(19) United States
(12) Patent Application Publication
(10) Pub. No.: US 2006/0172345 A1
(43) Pub. Date: Aug. 3, 2006

(54) Method for detecting low levels of a fusion protein

(75) Inventors: Frank Jakob Theodor Staal, Delft (NL); Jacobus Johannes Maria van Dongen, Nieuwerkerk aan den Ijssel (NL)

Correspondence Address: TRASK BRITT, P.O. BOX 2550, SALT LAKE CITY, UT, 84110, US

(73) Assignee: Erasmus University Medical Center Rotterdam, Rotterdam (NL)

(21) Appl. No.: 11/351,879

(22) Filed: Feb. 10, 2006

Related U.S. Application Data
(63) Continuation of application No. PCT/NL04/00562, filed on Aug. 11, 2004.

(30) Foreign Application Priority Data
Aug. 12, 2003 (EP) 03077529.0

(51) Int. Cl. G01N 33/542; G01N 33/53

(52) U.S. Cl. 435/7.9

(57) ABSTRACT

The invention relates to the detection of, among others, tumor-specific fusion proteins. Provided is a method for detecting a fusion protein in a sample, the fusion protein comprising an amino-terminal fragment and a carboxy-terminal fragment that each correspond to a native protein. The method comprises contacting the sample with at least one binding molecule specifically reactive with a part of the native protein that is not present in the fusion protein, under conditions that allow for the formation of a complex between at least one binding molecule and the native protein, removing the complex from the sample to deplete the sample of the native protein but not of the fusion protein; and detecting the fusion protein in the sample using at least one antibody probe directed against the fusion protein. Also provided is a diagnostic kit for carrying out a method of the invention.

### CROSS-REFERENCE TO RELATED APPLICATIONS

This application is a continuation of PCT International Patent Application No. PCT/NL2004/000562, filed on Aug. 11, 2004, designating the United States of America, and published in English, as PCT International Publication No. WO 2005/015235 A1 on Feb. 17, 2005, which application claims priority to European Patent Application No. 03077529.0 filed on Aug. 12, 2003, the contents of the entirety of each of which are hereby incorporated hereby by this reference.

### TECHNICAL FIELD

The invention relates generally to biotechnology and, more particularly, to the detection of, among others, tumor-specific fusion proteins. More specifically, the invention relates to techniques that indicate the presence of chromosomal translocations by detecting the presence of a fusion protein in a biological sample.

### BACKGROUND

In the diagnosis of various types of cancer, such as leukemias, lymphomas and solid tumors, chromosomal aberrations are frequently used for classification into prognostically relevant subgroups. Many of these chromosomal aberrations result in fusion genes, i.e., aberrantly coupled genes coupled via the upstream part of one gene to the downstream part of the other gene, or vice versa. Fusion genes can be transcribed into fusion gene transcripts and translated into fusion proteins. Generally, fusion proteins play an important role in the oncogenetic process. For example, approximately 35% of adult patients with acute lymphoblastic leukemia (“ALL”) and approximately 95% of patients with chronic myeloid leukemia (“CML”) have a specific chromosomal defect in their leukemic cells, i.e., a translocation between chromosomes 9 and 22 that creates the Philadelphia (Ph) chromosome. This translocation occurs at the site in the genome of a protein tyrosine kinase named ABL, creating the abnormal BCR-ABL fusion protein, a gene product of the in-frame fusion of the ABL gene with another gene called BCR. Generally, fusion proteins play an important role in the oncogenetic process. For example, the kinase activity of ABL in the BCR-ABL fusion protein is activated and deregulated, driving the uncontrolled cell growth observed in ALL and in CML.

When ALL is diagnosed in a patient, the total number of leukemia cells typically lies within the range of 10¹¹ to 10¹². Cytostatic or cytotoxic treatment induces complete remission in the majority of patients with lymphoid malignancies. Nevertheless, many of these patients relapse. Complete remission does not mean that the leukemic cells are totally eradicated from the body but that their level is beyond the sensitivity level of classical cytomorphologic methods. Since the detection limit of cytomorphological techniques is not lower than 1% to 5% malignant cells, this means that up to 10¹⁰ malignant cells can still remain in the patient, also indicated as the “minimal residual disease” (or “MRD”). Apparently, the current treatment protocols are not capable of killing all malignant cells in these relapsing patients, although they reached so-called complete remission according to cytomorphological criteria. Cytomorphological techniques can only provide superficial information about the effectiveness of treatment.

Detection of low numbers of residual malignant cells during early treatment response allows a precise assessment of the kinetics of tumor decrease during chemotherapy. It is now established that MRD information represents a powerful prognostic factor for final outcome. Detection of an increase of the MRD level enables anticipation of an impending relapse. Techniques with a high sensitivity to detect MRD are, therefore, crucial in order to obtain a better insight in the reduction of tumor mass during induction treatment and further eradication of the malignant cells during maintenance treatment.

Existing techniques for detecting chromosomal aberrations include traditional techniques such as cytogenetic analyses and biomolecular technology, such as Southern blotting or Fluorescent in situ hybridization (FISH) analysis. However, the detection sensitivity of cytogenetics, FISH analysis and Southern blotting is typically not lower than 1% to 5%, which makes them unsuitable for the detection of MRD.

PCR, although in essence well suited for rapid and massive diagnostic testing or even screening, allows only 0.1 to 4-5 kb of nucleic acid (e.g., DNA or RNA) to be analyzed routinely per PCR analysis, which greatly hampers rapid screening of vast stretches of chromosomes and breakpoint clusters or fusion regions within the chromosomes or their gene-products. An additional disadvantage of PCR is its inherent sensitivity to mismatched primers. Small alterations, which can always be present in the nucleic acid sequence of the gene fragment complementary to the primer, will make it impossible to operate the PCR with the wanted effect and may result in misdiagnosis and false-negative results. False-negative results especially render a PCR-based diagnostic test, albeit very specific, unsuitable for reliable diagnosis, and it goes without saying that only a reliable diagnosis of malignancies can contribute to an insight into the prognosis and to the design of an adequate therapy.

As indicated above, the majority of currently available techniques aimed at detecting specific chromosomal aberrations involve analysis at the chromosomal or nucleic acid (e.g., DNA or RNA) level. It is, however, also possible to detect a chromosomal aberration at the protein level.

Considerable effort has been undertaken in the field of leukemia diagnostics to develop a suitable technology for the immunological detection of a fusion protein resulting from a chromosomal aberration. Many of those studies focused on finding immunological reagents (antibodies) that are exclusively reactive with tumor-specific proteins and that do not, at the same time, give immunological detection of non-fusion proteins that are normally also produced by the body (see, for example, Nagasaki et al., J. Immun. Methods 162, 235-245, 1993, and van Denderen et al., J. Exp. Med. 1989 169(1):87-98). Usually, such antibodies cross-react with normal cellular proteins. Only when specific fusion points are known, it may be possible to select specific immunological reagents that react exclusively with the tumor-specific protein by selective binding to the fusion point epitope. However, the variation in fusion points is so large between patients (different breakpoints at the DNA level) and within patients (mRNA splice variants) that specific immunological detection only works in a few occasions, often solely on a patient-by-patient basis (see van Dongen et al., Leukemia 1999; 13:1901-1928). Furthermore, diagnostic tests involving immunodetection of the fusion point of a fusion protein have the great inherent disadvantage that they require specialized and well-equipped laboratories and trained and highly skilled personnel. Also, the above tests are typically only used in suspected cases of malignancies, and are not suitable for large-scale screening of leukemia patients for the presence of chromosomal aberrations.

The development of a rapid and more convenient immunological method allowing the exclusive detection of a fusion protein A-B was a major step forward in the area of diagnostic tests aimed at detecting a fusion protein (see, PCT International Patent Application PCT/NL98/00289). The reported method involves the application of a so-called catching antibody that recognizes one part of a fusion protein (part A) and a labeled detection antibody that recognizes another part of a fusion protein (part B). In such a system, a catching antibody is bound to a solid support layer, such as an ELISA plate or a dipstick (P. Berendes, “Recognition of tumor-specific proteins in human cancer,” Ph.D. Thesis, Erasmus University Rotterdam, ISBN 90-73436-36-2). A catching antibody can also be immobilized onto beads that can be analyzed by flow cytometry (see, for example, PCT International Patent Application PCT/NL01/00945). Following incubation of a bead-bound catching antibody with a cellular lysate suspected of containing a fusion protein, bound fusion protein is detected by a labeled detection antibody.

A bead-based catching/detection antibody system permits the exclusive detection of a fusion protein and is elegant and easy to perform, especially when using flow cytometry. Such a detection system also allows high-throughput diagnosis of many clinical samples. At the time of diagnosis of a malignancy involving a fusion protein, a diagnostic sample typically contains a relatively high number of malignant cells with a fusion protein compared to the number of normal cells which might contain native (non-fused) proteins. When ALL is diagnosed in a patient, the total number of leukemia cells is approximated to 10¹¹ to 10¹², of which approximately generally 25% to 98% contain a fusion gene encoding a fusion protein. A catching/detection antibody system is, therefore, suitable for the rapid screening for chromosomal aberrations that give rise to a fusion protein. Unfortunately, however, the sensitivity of a catching/detection antibody system often appears insufficient in detecting low frequencies (<1%) of malignant cells in a large background of normal cells. As an example, current catching/detection antibody systems are in general not adequate in detecting MRD during treatment follow-up. The diagnostic application of a catching/detection system is, therefore, essentially limited to the stage of initial diagnosis, when the relative frequency of malignant cells in bone marrow or blood is high (generally >10%). Taken together, there is a specific need for a method that allows, exclusively and conveniently, detection of a fusion protein in a sample, even if the sample contains only minimal amounts of the fusion protein.

It has now been recognized that, in current methods, a fusion protein and a non-fused native (i.e., wild-type) protein typically compete for specific binding to a probe that is used for the detection of the fusion protein. For example, a fusion protein A-B and a non-fused native protein A will compete for binding to a catching antibody, or other type of probe, specifically reactive with part A of the fusion protein. Likewise, a fusion protein A-B and a non-fused native protein B compete for specific binding to a catching antibody that is reactive with part B of the fusion protein. Thus, the sensitivity of a catching/detection antibody system for detecting a fusion protein comprising parts of at least two native proteins is largely determined by the proportion of a fusion protein present in a sample relative to the native non-fused proteins. When a native protein is in large excess of a fusion protein of interest, the native protein is likely to occupy most, if not all, binding sites of a catching antibody, thereby essentially preventing binding of a fusion protein. Furthermore, even if some fusion protein is efficiently caught by a catching antibody, the signal intensity indicative of binding of a labeled detection antibody may not be sufficient to yield a reliable test result.

### SUMMARY OF THE INVENTION

The invention provides the insight that the sensitivity of a method for detecting a fusion protein in a sample can be increased by depleting the sample of one or more native proteins prior to detecting the presence of a fusion protein, to enrich the sample for the fusion protein relative to one or more “competing” native proteins. Provided is a method to detect a fusion protein (e.g., resulting from a chromosomal translocation) in a sample via the detection of a fusion protein comprising an amino-terminal fragment and a carboxy-terminal fragment that are each corresponding to a different native protein, the method comprising: (i) contacting the sample with at least one binding molecule specifically reactive with a part of the native protein that is not present in the fusion protein, under conditions that allow the formation of a complex between at least one binding molecule and the native protein; (ii) removing the complex from the sample to essentially deplete the sample of the native protein but not the fusion protein; and (iii) detecting the fusion protein in the sample using at least one antibody directed against the fusion protein. The term “native protein” or “relevant native protein” as used herein refers to a non-fused, wild-type protein which can compete with a fusion protein of interest for binding to a probe or reagent that is used for the detection of a fusion protein, such as, for instance, a catching antibody. A native protein of the invention can adopt a native conformation (the three-dimensional structure of a protein when in a physiological environment) or a denatured conformation.

According to the invention, a fusion protein is derived from a fusion gene of a malignant cell or from an artificially produced fusion gene. In a fusion protein comprising an amino-terminal fragment and a carboxy-terminal fragment that are each corresponding to a native protein, the fragments are typically directly fused or connected to each other via a breakpoint fusion or fusion region such that these fragments together represent the whole fusion protein. The length or the size of a fragment, whether amino-terminal or carboxy-terminal, can vary. If the fusion region is located approximately halfway in a fusion protein, the fusion protein comprises an amino (N)-terminal fragment and a carboxy (C)-terminal fragment of approximately the same size. In other cases, the fusion region is located more closely towards either the C-terminal or N-terminal end of the fusion protein, such that the fusion protein consists of a large N-terminal fragment and a small C-terminal fragment or a large C-terminal fragment and a small N-terminal fragment, respectively. A fusion protein of the invention contains sufficient amino acids upstream and downstream from the fusion region to allow for (epitope) recognition by an antibody probe specifically reactive with parts of the fusion protein located at opposite sides of the fusion region or a binding fragment functionally equivalent thereto.

In a method of the invention, a sample is enriched for a fusion protein relative to one or more “competing” native proteins, resulting in increased binding of a fusion protein to a probe directed against the fusion protein. A method provided is particularly suitable to increase the sensitivity of a catching/detection antibody or sandwich system for the detection of a fusion protein. Thus, the invention provides a solution to a major problem encountered using existing detection methods wherein a native protein saturates or “consumes” a probe that is used to detect a fusion protein, such as a catching antibody, thereby preventing the binding of a fusion protein to the probe. Probe saturation with native protein reduces the sensitivity of detecting a fusion protein present in the same sample. By depleting a sample of a competing native protein according to the invention, the ratio of fusion protein/native protein is strongly increased, thus favoring binding of the detection probe and detection of the fusion protein. With a method provided herein, it is now possible to specifically detect the presence of a fusion protein in relatively rapid and simple fashion, even if the fusion protein is present in a sample in only a limited amount. For example, it provides for a catching/detection antibody system to detect a tumor-specific fusion protein in a sample comprising only a small population of fusion protein-positive malignant cells in a large background of normal cells, which contain a large number of competing, native proteins.

Furthermore, with a method according to the invention, it is now also possible to calculate the amount of native protein (as a measure for the amount of normal cells) relative to the amount of fusion protein (as a measure of the amount of malignant cells. The obtained ration of normal protein versus fusion protein can be used to determine the relative frequency of malignant cells, i.e., the level of MRD.

The invention provides the insight that the sensitivity of detecting the presence of a fusion protein in a cell, e.g., a chimeric oncoprotein, in a malignant cell, can be increased by depleting such a cell of at least one relevant native protein. It is possible to deplete more than one native protein prior to detection of a fusion protein. Needless to say, use of a method provided herein is most beneficial when detecting a fusion protein in a sample that also contains a significant amount of native proteins that can compete in binding to a probe that is used to catch a fusion protein. In one example, a method is provided for the detection of MRD by determining the presence of malignant cells in a sample, the method comprising detection of a tumor-specific fusion protein comprising parts of at least two native, non-tumor-specific proteins in a sample, the detection step being preceded by a “pre-clear” step wherein the sample is depleted of at least one of the native proteins, which would otherwise compete with the detection of the fusion protein.

In a method of the invention, at least one binding molecule is used to remove one or more native proteins from a sample. A sample comprises a biological sample such as a blood sample, serum sample, tissue sample, bone marrow, cerebrospinal fluid sample, biopsies and other samples that may contain one or more cells expressing a fusion protein encoded by a fusion gene. A sample is treated in such a manner that a binding molecule has sufficient access to a native protein. Because many cellular proteins are localized intracellularly, this is typically achieved by disrupting the membrane integrity of a cell. For example, a sample is treated in such a manner that it yields a cell lysate or a cell homogenate, for instance, obtained by mechanical rupture of cells or by exposing cells to a cell lysis buffer containing a detergent. A suitable binding molecule preferably comprises a protein or a polypeptide, like an antibody or an antibody fragment. However, other types of binding molecules are also instrumental in practicing a method provided. A specific antibody or fragment thereof can be obtained using various standard procedures known in the art, including methods to raise polyclonal antibodies in a laboratory animal, methods to obtain monoclonal antibodies using conventional hybridoma technology, and single-chain Fv antibody fragments (scFvs) using a phage display selection process. Such an antibody or antibody fragment is herein further referred to as “depleting antibody.”

For the design or development of a binding molecule that is reactive with a native protein but not with a fusion protein comprising a part of the native protein (such as a depleting antibody), it is, of course, crucial to know which part or fragment of the native protein is part of the fusion protein and which part is not. In other words, it is preferred that the fusion point or fusion region of a fusion protein to be detected is established. A method of the invention is advantageously used to detect a fusion protein that is the result of a known chromosomal aberration. In these cases, the resulting fusion genes and fusion proteins have often been characterized into detail. Well-known examples are the translocations resulting in BCR-ABL fusion genes found in >95% of cases of CML and in 30% of cases of adult ALL and the TEL-AML1 fusion genes which are found in 25 to 30% of childhood ALL cases. However, many more fusion genes and encoded fusion proteins are known to be involved in leukemia, such as E2A-PBX1, ETO-AML1 and PML-RARα.

It is to be understood that application of the present invention is not limited to the detection of leukemia; several other, non-leukemic diseases are characterized by the occurrence of fusion genes and fusion proteins. For example, the following ALK fusion genes can be observed in anaplastic large cell lymphoma (ALCL): the fusion gene NPM-ALK resulting from chromosome aberration t(2;5)(p23;q35); fusion gene TPM3-ALK resulting from chromosome aberration t(1;2)(q25;p23); fusion gene TFG-ALK resulting from t(2;3)(p23;q21) and fusion gene ATIC-ALK from inv(2)(p23q35). Depletion of native ALK protein from a sample according to the invention using a depleting antibody reactive with native ALK but not with the fusion protein will increase the sensitivity of a subsequent catching/detection assay. In another embodiment, the invention is advantageously applied to detect a chromosomal aberration observed in Ewing's sarcoma (EWS), such as t(11;22)(q24;q12) resulting in the EWS-FLI1 fusion protein, t(21;22)(q22;q12) resulting in the EWS-ERG fusion protein and t(7;22)(p22;q12) giving rise to the EWS-ETV1 fusion protein. Ewing's sarcoma (EWS)/Peripheral Primitive Neuroectodermal Tumors (PNET) of bone is a type of cancer usually found in children and young adults. The peak incidence is between ages 10 and 20; it is less common in children under 5 or in adults over 30. Ewing's sarcoma can occur in any bone in the body; the most common sites are the pelvis, thigh, lower leg, upper arm, and rib. Depletion of normal cellular EWS protein (and/or, optionally, depletion of the fusion partner gene such as FLI1, ERG or ETV1) in a pre-clear step according to the invention will increase the sensitivity of detecting a fusion protein related to Ewing's sarcoma.

Thus, the invention allows a person skilled in the art to apply and perform the invention without undue burden to the diagnosis or classification of any type of disease that is associated with the occurrence of a fusion gene that is translated into a fusion protein comprising an amino-terminal fragment and a carboxy-terminal fragment that are each corresponding to a native protein.

Based on the structure of a fusion protein, one can easily determine which part of the native protein is not present in the fusion protein. A binding molecule specifically reactive with this part, or a (polypeptide) stretch thereof, can be used to essentially remove the native protein, but not the fusion protein, from a sample. A person skilled in the art will recognize which steps to follow to obtain a binding molecule specifically reactive with the native (wild-type) protein but not with the fusion protein. In a preferred embodiment, such a specific binding molecule is an antibody or a binding fragment functionally equivalent thereto.

Methods of producing an antibody are known to those skilled in the art. For example, to obtain a polyclonal antibody, a laboratory animal is immunized with an immunogen. In a method of the invention, the immunogen is preferably a recombinant protein fragment or a synthetic peptide corresponding to the selected stretch or domain of the native protein. The animal's immune response is monitored by taking test bleeds and determining the titer of the reactivity. When appropriately high titers are obtained, blood is collected from the animal and antisera are prepared. Further fractionation of the antisera to enrich for antibodies reactive with the native protein can be done if desired. See, e.g., Harlow et al., Antibodies, A Laboratory Manual, Cold Spring Harbor Publications, New York (1988). Monoclonal antibodies can be obtained by various techniques known in the art, for example, by fusing spleen cells of immunized mice with a myeloma cell line by the addition of polyethylene glycol (PEG). Fused cells are cultured in a selection medium, for instance, medium containing a mixture of hypoxanthine, aminopterin and thymidine. Fused cells that survive in this selection medium are tested for the production of the desired antibody (for instance, by a solid-phase immunoassay such as ELISA) and, if positive, the cultures are cloned so that there is only one cell in each culture well. This produces a clone of cells from a single progenitor that is both immortal and a producer of monoclonal antibody. Antibodies obtained can be characterized using conventional immunodiagnostic techniques, for example, by Western blotting using lysates of cells expressing either the (recombinant) native protein or the (recombinant) fusion protein. Only those antibodies reactive with the native protein A, but not with the fusion protein A-B, are suitable for use as a depleting antibody in a method according to the invention.

According to the invention, a complex between a depleting antibody and a native protein is removed from a sample prior to detecting a fusion protein. In one embodiment, a method provided entails rapid and selective removal or precipitation of a native protein out of a cell lysate or homogenate via, for example, the formation of an antibody/antigen complex. Hereto, a sample is contacted with at least one binding molecule specifically reactive with a native protein under conditions that allow the formation of a complex between the binding molecule and the native protein. Various separation techniques can be used in a method according to the invention to remove a complex from a sample. Commonly used methods to precipitate an antibody/antigen complex out of a crude mixture include solid phase immunoprecipitation. In one embodiment, the complex is removed from the sample via the addition of solid beads or microspheres capable of binding the complex, to form a suspension of beads in an essentially liquid sample. According to the invention, a suitable bead comprises a Sepharose bead, or a polystyrene bead, or any kind of solid particle that allows the simple removal of a bead-bound complex from a sample. The advantage of solid beads is that they can be simply pelleted from a sample, typically by centrifugation of the suspension. Alternatively, a pre-cleared sample is obtained by filtration of the suspension. In one embodiment of the invention, a sample is contacted with a depleting antibody to form a complex with a native protein and an immunoglobulin-binding protein immobilized onto a bead is used to remove the complex from the sample. Various types of immunoglobulin-binding proteins exist that can be used suitably (Antibodies, a Laboratory Manual, E. Harlow and D. Lane (1988)). These include protein A, protein G and bacterial cell ghosts of Staphylococcus aureus. S. aureus has a special protein on its surface called Protein A, which is a versatile, broad range IgG-binding reagent. Protein A binds the Fc portion of antibodies (IgG class) without disturbing their binding of antigen. Antibodies from many mammalian species will generally react with Protein A. In another embodiment, GammaBind™ G Type 2 or Protein G is used to remove a complex of a depletion antibody and a native protein from a sample. GammaBind™ G Type 2 and Protein G are recombinant engineered forms of streptococcal protein G. Both proteins can be produced in Escherichia coli. They bind to the constant region of many types of immunoglobulin G, and are widely used to detect, quantify and purify IgG antibodies and antigen/antibody complexes. GammaBind™ G Type 2 and Protein G are the most universally applicable antibody binding proteins available. Compared with Protein A, they bind tightly and specifically to antibodies from many different species. For example, GammaBind™ G Type 2 binds better to mouse and rat IgG; GammaBind™ G Type 2 and Protein G bind selectively to all subclasses from human IgG. Bacterial ghosts, immunoglobulin-binding proteins and beads coated with immunoglobulin-binding proteins can be commercially obtained from various suppliers, such as Pharmacia Biotech AB, Sigma Aldrich or Pierce. In a method provided herein, following the formation of a complex between a native protein and a depleting antibody, bacterial ghosts or coated beads are mixed with the cell sample or cell homogenate, during which time the immunoglobulin-binding protein attaches to the depleting antibody. A brief and simple centrifugation or filtration step can separate the bacterial ghosts or beads with attached depleting antibody/native protein complex from the sample.

In a preferred embodiment, however, a binding molecule (preferably a depleting antibody) is directly coupled to or immobilized onto a bead or other solid particle. Display of biochemical reagents on synthetic microbeads is a prevailing trend in the development of bioanalytical assays. Commercial sources (e.g., Spherotech, Ill.; Bangs, Ind.; Polysciences, Pa.; Molecular Probes, Oreg.) for bead-based display systems (e.g., covalent coupling, biotin-streptavidin, His-tag) are currently available. Use of a depleting antibody that is directly conjugated to a bead does not require the intermittent use of an immunoglobulin-binding protein and will, in general, result in increased specificity. Following incubation for a sufficient time period, beads with attached depleting antibody complexed to native protein can be removed from a sample, such as a lysate or homogenate. Removal of beads from a sample is easily and rapidly achieved by, for instance, centrifugation, resulting in a sample that is essentially depleted of a native protein that would otherwise compete with a fusion protein for binding to a probe directed against the fusion protein. Because this depletion step results in a sample that is enriched for a fusion protein relative to the “competing” native, non-fused protein, the probability that a fusion protein will bind to such a probe, for instance, a catching antibody, is significantly increased. For example, a method provided now allows detection of a fusion protein using a catching/detection system with increased sensitivity.

In another embodiment of the invention, a depletion antibody or any other type of suitable binding molecule, is conjugated to a magnetic bead. This allows the removal of a bead-bound complex from a sample using immunomagnetic separation (IMS). IMS is a technique that involves adding magnetic beads coated with a binding molecule, which is specifically reactive with an antigen of interest, to a cellular lysate. The beads and lysate are mixed, during which time a complex is formed between a depleting antibody or other binding molecule and a native protein to form an antigen/antibody complex. After a certain incubation period, a magnetic field is applied to separate the magnetic beads or magnetic particles with attached complex from other substances, leaving behind a depleted or “pre-cleared” sample. Suitable magnetic beads are, for example, those marketed by Spherotec Inc. (www.spherotech.com) under the brand name SPHERO magnetic particles. They are prepared by coating a layer of magnetite and polystyrene onto monodispersed (uniformly sized) polystyrene core particles (see, U.S. Pat. No. 5,091,206). As a result, the particles are spherical in shape and paramagnetic in nature. They are also very uniform in size. The magnetite contents of these magnetic particles can be adjusted but, in general, it represents about 10% to 15%. The magnetic particles can be easily magnetically separated from a suspension. These particles become non-magnetic when removed from a magnet and do not retain any detectable magnetism even after repeated exposure to a strong magnetic field. The magnetic particles can be used for cell separation, affinity purification, DNA probe assays, magnetic particle EIA, etc. Various sizes of beads, be it Sepharose beads, polystyrene beads, latex beads, magnetic beads or the like, are commercially available. In a preferred embodiment, beads with a relatively small diameter are used because, for a certain volume of beads, small beads have a larger surface area compared to large beads. The larger the bead surface, the more binding molecules can be immobilized onto a bead and the more efficiently a native protein can be removed from a sample per volume of beads. This is especially advantageous when removing a native protein from a sample with a small sample volume.

As disclosed herein, the invention solves the problem that a catching/detection antibody system, although rapid and easy to perform, is often not sensitive enough to detect the presence of low amounts of a fusion protein in a sample. A method is provided to detect a fusion protein comprising parts of two different native proteins with increased sensitivity by including a pre-treatment step to obtain a sample that is enriched in a fusion protein of interest relative to one or both of the native proteins. This enriched or pre-cleared sample is processed further to detect the presence of a fusion protein. In theory, a fusion protein A-B present in a sample that is completely depleted from native protein A using a method provided, can be detected using a single antibody (or functional equivalent thereof) directed against part A of the A-B fusion protein. However, as it is very difficult in practice to obtain a pre-cleared sample that does not contain any native protein A, use of a single A-specific antibody may not always give the desired detection specificity. Thus, when aiming for the exclusive detection of an A-B fusion protein, it is preferred to use at least one additional antibody that is directed against part B of the fusion protein. For example, a fusion protein is detected using a set of at least a first and a second antibody, each antibody capable of recognizing a binding site positioned at opposite sides of the fusion region of the fusion protein. In a preferred embodiment of the invention, the invention provides a method wherein a fusion protein derived from a fusion gene is detected by flow cytometric detection or by any other convenient way to measure bead-associated fluorescence using at least two antibody probes, wherein at least one is directed against a protein fragment comprising the amino-terminal fragment of the fusion protein, and at least one other is directed against a protein fragment comprising the carboxy-terminal fragment of the fusion protein.

The use of microspheres, beads, or other particles as solid supports for antigen-antibody reactions in order to detect antigens or antibodies in a sample is particularly attractive when linked to flow cytometry (see, e.g., U.S. Pat. No. 6,159,748). In a preferred embodiment, an antibody probe is coupled to a bead that allows the detection of a fusion protein via flow cytometry. Flow cytometers have the capacity to detect particle size differences and are highly sensitive fluorescence detectors. Microspheres can be sized by forward angle light scatter (FALS) or electronic volume. Used in conjunction with right angle light scatter (RALS), a flow cytometer can distinguish between single and aggregated particles.

A method according to the invention is very attractive for the detection of a tumor-specific protein comprising an amino-terminal fragment and a carboxy-terminal fragment that are each corresponding to a different non-tumor-specific protein. The tumor-specific fusion protein is, for example, a result of a Philadelphia chromosome aberration, creating the abnormal BCR-ABL protein. Prior to detecting, one or both of the native proteins BCR and ABL are depleted from a sample to be analyzed by using at least one binding molecule specifically reactive with a fragment of the ABL or BCR protein, but not with the BCR-ABL fusion protein. Following removal of the complex comprising BCR and/or ABL, the BCR-ABL fusion protein is detected, preferably using a set of at least a first and a second antibody, wherein each antibody recognizes a binding site positioned at opposite sides of the fusion region of the fusion protein. The fusion protein can be detected using a catching/detection system, for instance, using flow cytometry, ELISA, or a dipstick method (see also the Example below).

A method of the invention is especially suitable to detect very low numbers of cells expressing a fusion protein (<1% such as encountered in MRD). However, it is, of course, also advantageously used to increase the sensitivity of detection methods that do not require such low detection limits, such as those used for the initial diagnosis of a disease that is correlated with the presence of a fusion protein. Also, in these cases, removal of one or more native proteins prior to detecting a fusion protein will result in enhanced sensitivity. At the stage of diagnosis, it is often desired to investigate the occurrence of different fusion gene proteins, preferably simultaneously in one tube. This is not because a particular malignancy will have more than one fusion gene protein, but because it is convenient to have a single test tube for detection of several well-established fusion gene proteins within one disease category, e.g., acute myeloid leukemia (AML) or precursor-B-ALL (Table 1).

TABLE 1
Examples of combined detection of several
fusion proteins in a single tube.

| Disease | Chromosomal | | Relative frequency | |
|---|---|---|---|---|
| category | aberration | Fusion protein | children | adults |
| AML | t(8; 21) (q22; q22) | AML1-ETO | 10%-14% | 6%-8% |
| | t(15; 17) (q22; q21) | PML-RARA | 8%-10% | 5%-15% |
| | inv(16) (p13; q22) | CBFB-MYH11 | 5%-7% | 5%-6% |
| Precursor | t(1; 19) (q23; p13) | E2A-PBX1 | 5%-8% | 3%-4% |
| B-ALL | t(4; 11) (q21; q23) | MLL-AF4 | 3%-5% | 3%-4% |
| | t(9; 22) (q34; q11) | BCR-ABL | 3%-6% | 25%-40% |
| | t(12; 21) (p13; q22) | TEL-AML1 | 25%-30% | 0%-2% |

In a preferred embodiment, a method provided herein comprises the flow cytometric detection of different types of fusion proteins, preferably in a single tube assay, by using different bead-bound catching antibodies against one part of the different fusion proteins and the relevant corresponding detection antibodies against the other part of the fusion proteins. By combining FALS and fluorescence, it is practical to use beads of several different sizes, each bead coated with a different catching antibody, for the simultaneous detection of multiple fusion proteins. Microspheres can be coated with proteins passively or covalently depending on their chemical makeup. Based on different flow cytometric characteristics of the beads (e.g., size, fluorochrome color, intensity of fluorochrome staining, or side scatter characteristics), multiple fusion proteins can be specifically detected in the same assay. This also includes the detection of fusion proteins from various variant translocations of the same target gene as well as fusion proteins from translocations with variant breakpoints. An essential part of a method according to the invention for the detection of one or more fusion proteins, be it using a catching/detection system or any other kind of detection procedure, lies in the fact that fusion protein detection is preceded by the depletion of one or more relevant native, non-fused proteins. This can be achieved using one or more binding molecules, such as depletion antibodies, that can specifically recognize and bind to a native protein to form a complex. Different binding proteins may be attached to different single solid support surfaces, like a bead. Alternatively, multiple binding partners specifically reactive with different native proteins are coupled to the same bead such that only a single bead can be used to simultaneously deplete a sample of multiple native proteins. This is especially of interest when a method according to the invention is used to investigate the occurrence of different fusion proteins simultaneously in one tube, e.g., for the diagnosis of a malignancy within one disease category, particularly when using patient samples with a low tumor load (e.g., less than 5%) at diagnosis.

Thus, a method of the invention allows for the simultaneous detection of a first fusion protein A-B, comprising the N-terminal fragment of the native protein A and the C-terminal fragment of the native protein B, and a second fusion protein C-D, comprising the N-terminal fragment of the native protein C and the C-terminal fragment of the native protein D, in a single tube assay with a high specificity and improved sensitivity compared to existing detection methods. In this specific example, a patient sample with a low tumor load is, for example, contacted with a depletion antibody specifically reactive with the C-terminal fragment of protein A (anti-A) and with a depletion antibody specifically reactive with the N-terminal fragment of protein D (anti-D), to deplete the sample from native proteins A and D, respectively. The anti-A and anti-D depletion antibodies are preferably immobilized onto a small bead; even more preferred, onto the same bead for reasons of efficiency and ease of use. A simple centrifugation step is, in general, sufficient to separate the complex, in this specific example consisting of beads/anti-A/protein A/anti-D/protein D, from the sample. As a result, a sample is obtained that is essentially depleted of native proteins A and D. This will improve the likelihood that the fusion proteins A-B and C-D will be recognized by their specific probes, e.g., a catching antibody specifically reactive with the N-terminal fragment of fusion protein A-B and the C-terminal fragment of fusion protein C-D.

In addition to the limited sensitivity, current methods for detecting a fusion protein in a sample face the common problem that the possibility to quantify the amount of fusion protein detected is limited. Especially when applying these methods as diagnostic tools, it is highly desirable to quantify, or at least semi-quantify, a fusion protein. If possible at all, a detection signal, such as fluorescence intensity, which indicates the presence of a fusion protein, can be expressed relative to an internal control factor assumed to be constant, irrespective of whether or not a fusion protein is present. Such a factor can, for instance, be the number of cells analyzed or the total protein content of a sample. This relative value can be used for the purpose of comparison between different samples, for example, a positive and a negative control sample. It would be far more informative to directly relate the presence of a fusion protein comprising parts of two native proteins to one, or even both, non-fused native protein(s).

Thus far, none of the existing detection methods allows for such quantification. Importantly, the present invention now also provides a simple and straightforward solution to this problem. A complex containing one or more depleted native protein(s) that is removed from a sample in a method of the invention is preferably analyzed further to indicate the amount of native protein(s) depleted from the sample. In a preferred embodiment, this analysis comprises the use of at least one reagent capable of specifically binding to one or more components of the depleted or isolated complex, followed by determining the binding of the reagent to the complex as an indication of the amount of native protein that was depleted from the sample. A reagent preferably comprises a binding partner, like an antibody or a functionally equivalent fragment thereof, capable of binding a depleted native protein. In a preferred embodiment, a depleted complex is detected using a reagent directed against a depleted native protein, at a region that is distinct from the site of interaction between the native protein and a depletion antibody. More preferred, a reagent for detecting a depleted native protein reacts with that part of the native protein that is also present in the fusion protein. For example, a depleted protein A is advantageously detected using the “catching antibody” used for the detection of the A-B fusion protein as a reagent. A reagent is preferably provided with a reporter molecule or label, like a fluorochrome, allowing detection of a complex via the binding of a labeled reagent to a depleted protein.

Detection of a complex to determine the amount of native protein depleted from a sample is advantageously carried out using flow cytometry. In one embodiment, a depletion antibody is conjugated to a bead. Suitable beads comprise those discussed above for detection of a fusion protein, including magnetic beads compatible with flow cytometry.

When the amount of depleted native protein is known, this information can be used as an indication of the amount of cell material or total protein content. In other words, the depleted protein can serve as a kind of “internal control” in the diagnostic test. When relating the presence (expression) of a protein in an isolated complex to the amount of cell material or to the number of cells, it is preferred that the isolated complex contains a sufficient amount of the native protein to be detected by a reagent. Thus, a native protein to be depleted and analyzed further as a measure of the amount of cell material preferably comprises a protein that is relatively ubiquitous in the cells. In addition, it is important that the expression of the native protein is not subject to large variations, for example, between individual cells or under different cellular conditions. In a preferred embodiment, the native protein to be depleted and analyzed further is an abundant protein with a stable expression, such as a housekeeping protein. For example, when detecting the BCR-ABL fusion protein in a sample, it is advantageous to deplete and subsequently detect the native ABL protein because ABL is an abundant protein. However, a fusion protein does not always comprise a fragment of an abundant native protein. For example, in the E2A-PBX1 fusion protein, the N-terminal transactivation domain of the basic helix-loop-helix (bHLH) transcription factor, E2A, is joined to the majority of the pre-B-cell leukemia transcription factor 1 (PBX1). Transcription factors are typically expressed at a low level and in a cell-type specific fashion. Thus, in these cases, it is not preferred to use any of the depleted native proteins as an internal control. Instead, it is advantageous to use an additional antibody probe that simultaneously depletes a sample of an abundant protein. To illustrate this further, in the situation depicted above, a sample, for instance, a cell lysate, is contacted with a bead that is coated with two antibodies: one antibody reactive with native E2A but not with the E2A-PBX1 fusion protein, to deplete the sample of competing E2A protein and one antibody reactive with a housekeeping protein (such as ABL) to deplete the sample at the same time of a housekeeping protein that can serve as internal control.

According to an aspect of the invention, semiquantification of a depleted native protein can be performed by constructing a standard curve relating a measured quantity (e.g., fluorescence) to “known” amounts of native protein. Preferably, “known” samples contain the native protein in amounts chosen to span the concentration range of native proteins that are expected in the “unknown” samples (e.g., using serial dilutions of normal cells). Such a standard curve can then be used to determine concentrations of the depleted native protein substance in an “unknown” sample (or a dilution thereof) containing the depleted complex.

The invention provides a method as discussed above using probes that can be labeled or conjugated with reporter molecules, such as biotin, digyoxigenin, enzymes such as peroxidase, alkaline phosphatase, or other reporter molecules or reporter particles, such as beads, known in the art. The invention further provides a diagnostic kit comprising all the means, such as binding molecules, conjugated beads, labeled probes or reagents or substrate or instructions, necessary to carry out the method according to the invention. Methods or diagnostic kits provided by the invention are preferably used to detect chromosomal aberrations found with certain types of cancer, for example, with leukemia, be it in the detection of residual cancer in patients or the screening for cancer in larger populations as a whole.

Strengths of a method according to the invention are: 1) the ability to simultaneously, but discretely, analyze multiple fusion proteins (most relevant at the time of diagnosis); 2) the simplicity of binding proteins to microspheres; 3) the ability of flow cytometry to detect small particle size differences; and 4) the exquisite sensitivity of flow cytometry as a detector of different wavelengths of fluorescence, simultaneously. Available auto-sampling systems make it even more appealing in this regard.

A method of the invention is advantageously used in diagnostic testing of biological samples, such as blood samples, serum samples, samples of cells, tissue samples, cerebrospinal fluid, bone marrow, or biopsies for chromosomal aberrations. The invention provides a method to be used in diagnostic testing where both a high sensitivity as well as a high specificity is required. A method provided now has the ability to both diagnose a malignancy and closely monitor MRD during follow-up of patients with various types of cancer that involve chromosomal translocations, inversions or deletions that give rise to a fusion gene. Use of a method of the invention is provided before, during and after treatment of a disease to evaluate the effectiveness of the treatment.

### DETAILED DESCRIPTION OF THE INVENTION

### Example

Detection of BCR-ABL Fusion Protein in Pre-Cleared Cell Lysates

This example illustrates how normal (i.e., native) ABL can be removed from a sample prior to detection in a catching/detection antibody assay to improve the sensitivity of detecting the BCR-ABL fusion protein in an MRD assay. Such a “pre-clear” step can be performed with an anti-ABL antibody against the N-terminal part of ABL. All normal ABL expressed in the cells will be cleared and only the ABL fragment that is present in the BCR-ABL fusion protein will bind to the detection beads carrying anti-ABL catching antibody against the C-terminal part of ABL. BCR-ABL bound to the beads is then detected using an anti-BCR detection antibody conjugated to a fluorescent label.

Materials

1) Leukemic cells obtained from an MRD patient cultured in medium
2) Phosphate-buffered saline (PBS)/2% fetal calf serum (FCS): add 2 ml FCS (heat-inactivated) to 100 ml PBS pH 7.8; store at 4° C.
3) Radioimmunoprecipitation (RIPA)-buffer (without sodium deoxycholate): Preparation of 50 ml of RIPA-buffer:
   50 mM Tris-HCl: use 2.5 ml of a 1 M stock Tris-HCl
   150 mM NaCl: use 1.5 ml of a 5 M stock NaCl
   1% NP40: use 0.5 ml of undiluted Nonidet P40 (BDH, #56009 2L)
   0.1% SDS: use 0.5 ml of a 10% stock SDS
   Add 45 ml milli-Q
   Store at 4° C.
4) Protease inhibitor cocktail, Sigma, #P2714: dissolve the powder in 2 ml milli-Q to prepare a 50× concentrated solution; store at 4° C.
5) Pre-clear beads: Polystyrene beads (>10 μm) coated with depleting antibody directed against the N-terminus of ABL.
6) Detection beads: Luminex, PolyScience, or Molecular Probe beads, coated with catching antibody directed against the C-terminus of ABL.
7) Detection antibody: either FITC- or biotin-conjugated antibody directed against the N-terminus of BCR.
8) Streptavidin (SA)-PE, Caltag Laboratories, #SA1004-1.
   Methods
   Preparation of Cell Lysates
1) Transfer the number of cells needed and exactly calculated to a fresh tube and wash cells twice with PBS. Minimally, 1×10⁶ cells are typically required for the analysis of a fusion protein. However, more cells, e.g., up to 50×10⁶ cells, may be needed if the MRD level (and, therefore, the levels of aberrant fusion protein) is expected to be very low.
2) In the meantime, calculate the volume of RIPA-buffer needed to make cell lysates: 50 μl. RIPA-buffer per 500,000 cells. Transfer the RIPA-buffer to a 15 ml tube and add 50× concentrated protease inhibitor cocktail (20 μl protease inhibitor cocktail per 1 ml RIPA-buffer).
3) Resuspend the cell pellets in the appropriate amount of RIPA-buffer (5×10⁶/ml) containing protease inhibitors and transfer the solutions to fresh Eppendorf tubes.
4) Incubate on ice for 30 minutes.
5) Centrifuge 10 minutes at 10,000×g, 4° C.
6) Transfer the supernatant (cell lysate) to a fresh Eppendorf tube.
   Pre-Clear of Cell Lysate
7) Add 1×10⁶ pre-clear beads to 100 μl of cell lysate sample.
8) Incubate on ice for 30 minutes.
9) Spin at 10,000×g for 3 minutes and transfer the supernatant to a new Eppendorf tube.
10) Repeat step 9.
   Detection of Fusion Protein
11) Add to one test-tube:
   100 μl of pre-cleared cell lysate sample
   10,000 catching beads in 10 μl PBS+2% FCS
   1-2 μg of conjugated detection antibody in 25 μl PBS+2% FCS
12) Incubate on ice for 30 minutes.
13) Wash the beads with 500 μl PBS/2% FCS and spin for 1 minute at 14,000×g. Remove supernatant and repeat this step.

When a biotin-labeled detection antibody is used, continue with steps 14 to 18.

When a FITC-labeled detection antibody is used, continue with steps 17 and 18.
14) Dilute SA-PE 200 times in PBS/2% FCS and resuspend the beads in 20 μl of the diluted SA-PE per tube.
15) Incubate for 10 minutes at room temperature.
16) Wash the beads with 500 μl PBS/2% FCS and spin for 1 minute at 14,000×g. Remove supernatant and repeat this step.
17) After the second wash, resuspend the beads in 100 to 150 μl PBS/2% FCS per tube and transfer to FACS-tubes.
18) Measure the amount of label on the beads with a flow cytometer. The ratio of the amount of label and the initial number of cells is a measure of MRD level.

1. A method for detecting a fusion protein in a sample, said fusion protein comprising an amino-terminal fragment and a carboxy-terminal fragment that each correspond to a native protein, said method comprising:
contacting the sample with at least one binding molecule specifically reactive with a part of the native protein that is not present in the fusion protein, under conditions that allow for the formation of a complex between said at least one binding molecule and said native protein;
removing said complex from the sample to deplete the sample of native protein; and
detecting said fusion protein in the sample using at least one antibody or antibody fragment directed against said fusion protein.

2. The method according to claim 1, wherein said at least one binding molecule is an antibody or a binding fragment.

3. The method according to claim 1, wherein said at least one binding molecule is conjugated to a solid particle or a bead.

4. The method according to claim 3, wherein said complex is removed from the sample by centrifugation or by magnetic separation.

5. The method according to claim 1, wherein said fusion protein is detected using a set of at least a first antibody and a second antibody, each antibody capable of recognizing a binding site positioned at opposite sides of the fusion region of said fusion protein.

6. The method according to claim 1, wherein at least one antibody is labeled with a fluorochrome.

7. The method according to claim 1, wherein at least one antibody is coupled to a bead.

8. The method according to claim 1, wherein said fusion protein is detected via flow cytometry.

9. The method according to claim 1, further comprising:
after the step of removing said complex from the sample, detecting said complex using at least one reagent capable of specifically binding to said complex and determining the binding of said at least one reagent to said complex as an indication of the amount of native protein depleted from the sample.

10. The method according to claim 9, wherein said native protein is an abundant protein or a housekeeping protein.

11. The method according to claim 1, wherein said fusion protein comprises an amino-terminal fragment and a carboxy-terminal fragment that each corresponds to a different non-tumor-specific protein.

12. The method according to claim 11, wherein said fusion protein is a result of a Philadelphia chromosome aberration.

13. The method according to claim 12, wherein the amino-terminal fragment of said fusion protein corresponds to the ABL or BCR protein whereas the carboxy-terminal fragment of said fusion protein corresponds to the BCR or ABL protein, respectively.

14. The method according to claim 13, wherein said at least one binding molecule, is specifically reactive with a fragment of the ABL or BCR protein, but not with the fusion protein.

15. A diagnostic kit for carrying out the method according to claim 1, said diagnostic kit comprising:
a binding molecule and
at least one probe.

16. A method for detecting a fusion protein in a sample, wherein said fusion protein comprises an amino-terminal fragment and a carboxy-terminal fragment that each correspond to a native protein, said method comprising:
contacting the sample with means for specifically reacting with a part of the native protein not present in the fusion protein, under conditions that allow for the formation of a complex between the means for specifically reacting and the native protein;
removing complex thus formed from the sample so as to deplete native protein from the sample; and
detecting the fusion protein in the sample using at least one antibody or antibody fragment directed against the fusion protein.

17. The method according to claim 16, wherein said means for specifically reacting with a part of the native protein not present in the fusion protein is an antibody or a binding fragment.

18. The method according to claim 17, wherein said means for specifically reacting with a part of the native protein not present in the fusion protein is conjugated to a solid particle or a bead.

19. The method according to claim 18, wherein said complex is removed from the sample by centrifugation and/or magnetic separation.
